(19) United States
(12) Patent Application Publication
(10) Pub. No.: US 2009/0155220 A1
(43) Pub. Date: Jun. 18, 2009

(54) Use of Bone-Marrow Derived Stem Cells to Treat Ischemia

(75) Inventor: Douglas W. Losordo, Chicago, IL (US)

Correspondence Address: EDWARDS ANGELL PALMER & DODGE LLP, P.O. BOX 55874, BOSTON, MA, 02205, US

(73) Assignee: Caritas St. Elizabeth's Medical Center of Boston

(21) Appl. No.: 12/090,706

(22) PCT Filed: Oct. 20, 2006
(86) PCT No.: PCT/US06/41101

§ 371 Date: Oct. 8, 2008

Related U.S. Application Data
(60) Provisional application No. 60/728,509, filed on Oct. 20, 2005.

(51) Int. Cl. A61K 35/12

(52) U.S. Cl. 424/93.7

(57) ABSTRACT

Disclosed are cellular compositions and methods for preventing, treating or reducing the severity of tissue ischemia, particularly limb ischemia in a mammal. One inventive method includes administering a therapeutically effective amount of a cellular composition comprising a novel isolated multi-potent human bone marrow-derived stem cell (BMSC) having undetectable or negligible levels of markers of other known stem cells isolated from bone marrow. These cells can be expanded in vitro and formulated into cellular compositions and grafts capable of differentiating into components of functional new blood vessels when directly administered into ischemic limb tissue.

### CROSS-REFERENCE TO RELATED APPLICATION

The present application claims priority from U.S. Provisional Patent Application No. 60/728,509 entitled Use of bone marrow derived stein cells to treat limb ischemia as filed on Oct. 20, 2005, the disclosure of which is incorporated by reference.

### STATEMENT AS TO FEDERALLY SUPPORTED RESEARCH

The present invention was made with United States government support under National Institutes of Health (NIH) grant numbers HL53354, HL63414, HL63695, and HL-66957. Accordingly, the United States government may have certain rights to the invention.

### FIELD OF THE INVENTION

The invention generally relates to compositions and methods for treatment of ischemic disorders, particularly ischemic disorders of the extremities associated with compromised blood flow such as limb ischemia. More particularly, the invention features the use of a bone marrow-derived stem cells for cell therapy for ischemic disorders.

### BACKGROUND OF THE INVENTION

Tissue ischemia is a result of localize hypoxia, an insufficient supply of oxygenated blood, being delivered to tissues, organs, and limbs. Ischemia can result from progressive deterioration of circulation from conditions such as atherosclerosis or diabetes, or from more acute conditions or insults including tissue transplant, limb reattachment, or treatment with some therapeutic agents. Untreated, ischemia can result in cell death by necrosis and apoptosis.

Chronic critical limb ischemia (distal ischemia) is a debilitating condition characterized by pain at rest, nonhealing wounds and gangrene. The development of chronic critical limb ischemia usually requires multiple sites of arterial obstruction that severely reduce blood flow to the tissues (Haimovici, 1967; Mavor, 1956). The condition is the end result of arterial occlusive disease, most commonly atherosclerosis. Atherosclerosis can occur in association with hypertension, hypercholesterolemia, cigarette smoking and diabetes (Gordon et al., 1972; Brown et al., 1972). Less frequent causes of chronic critical limb ischemia include Buerger's disease, or thromboangiitis obliterans, and some forms of arteritis (DeBakey et al., 1964).

Ischemic rest pain is classically described as a burning pain in the ball of the foot and toes that is worse at night when the patient is in bed. The pain is exacerbated by the recumbent position because of the loss of gravity-assisted flow to the foot. Ischemic rest pain is located in the foot, where tissue is farthest from the heart and distal to the arterial occlusions (Santilli et al., 1996). Patients with ischemic rest pain often need to dangle their legs over the side of the bed or sleep in a recliner to regain gravity-augmented blood flow and relieve the pain. Patients who keep their legs in a dependent position for comfort often present with considerable edema of the feet and ankles.

Nonhealing wounds are usually found in areas of foot trauma caused by improperly fitting shoes or an injury. A wound is generally considered to be nonhealing if it fails to respond to a four- to 12-week trial of conservative therapy such as regular dressing changes, avoidance of trauma, treatment of infection and debridement of necrotic tissue.

Gangrene is usually found on the toes. It develops when the blood supply is so low that spontaneous necrosis occurs in the most poorly perfused tissues.

A number of physical findings and objective hemodynamic parameters can be used to substantiate a diagnosis of chronic limb ischemia. Typical physical findings include absent or diminished pedal pulses, shiny smooth skin of the feet and legs, and muscle wasting of the calves. An objective measurement of blood flow is accomplished with the use of a hand-held Doppler probe and a blood pressure cuff (Santilli et al., 1996). The cuff is inflated until the pulse distal to the cuff is no longer heard by Doppler. The cuff is then slowly deflated until the pulse is again detected. This measurement is recorded as the systolic pressure. An ankle systolic pressure of 50 mm Hg or less or a toe systolic pressure of 30 mm Hg or less suggests the presence of critical limb ischemia. Another widely used parameter is the ankle-brachial index, which is a ratio of the systolic pressure at the dorsalis pedis or posterior tibial artery divided by the systolic pressure at the brachial artery. Patients with critical limb ischemia usually have an ankle-brachial index of 0.4 or less (Cutajar et al., 1973; Ouriel et al., 1982).

Many patients with critical limb ischemia have a stable or slowly progressive presentation. Review of data reveals that patients with chronic critical limb ischemia have a three-year limb loss rate of about 40 percent (Cheshire et al., 1992; Eklund et al., 1982).

Medical intervention for chronic limb ischemia can range from conservative therapy to revascularization or in some cases amputation. Progressive gangrene, rapidly enlarging wounds or continuous ischemic rest pain can signify a threat to the limb and suggest the need for revascularization in patients without prohibitive operative risks. Bypass grafts are usually required because of the multilevel and distal nature of the arterial narrowing in critical limb ischemia. Compared with amputation, revascularization is more cost-effective and is associated with better perioperative morbidity and mortality.

While carefully designed conservative therapy can benefit many patients with critical limb ischemia, the severe nature of their disease often leads to consideration of operative intervention. Surgical interventions include revascularization or amputation. If the patient is willing to undergo revascularization and is an acceptable operative candidate, arteriography is often performed for further evaluation and planning of revascularization. At some centers, magnetic resonance angiography is used as an alternative or supplement to arteriography to minimize the risk of dye exposure (Welch, 1997). Limb preservation by means of revascularization, as in chronic limb ischemia, results in better outcomes. It is widely recognized that limb preservation should be the goal for most patients with chronic critical limb ischemia (Cheshire et al., 1992)

The feasibility of revascularization is determined by the arteriographic findings as well as the availability of a bypass conduit. Angioplasty or stent placement, or both, is most successful with short, proximal lesions, but is unlikely to be the only treatment necessary in the setting of critical limb ischemia because of the multilevel nature of the arterial occlusive disease. The ideal bypass conduit is the greater saphenous vein, but other conduits include the lesser saphenous veins, the arm veins or a prosthetic conduit. In most surgical series, three-year bypass patency rates of calf arteries range from 40 percent for prosthetic bypasses to 85 percent for saphenous vein bypasses (Taylor et al., 1987; Taylor et al., 1990; Donaldson et al., 1991). In comparison, studies of conservative therapy have demonstrated a 25 to 49 percent success rate with nonhealing wounds and a 50 to 80 percent rate of improvement in ischemic rest pain (Cheshire et al., 1992; Eklund et al., 1982). Despite these advances, there remains a serious need for improved therapies for limb preservation and quality of life for patients with limb ishchemia.

Diabetes is a particularly important risk factor for critical limb ischemia, as well as ischemia for other tissues, because it is frequently associated with severe peripheral arterial disease. Atherosclerosis develops at a younger age in patients with diabetes and progresses rapidly. Moreover, atherosclerosis affects more distal vessels in patients with diabetes the profunda femoris, popliteal and tibial arteries of the legs are frequently affected, while the aorta and iliac arteries are minimally narrowed. These distal lesions are less amenable to revascularization than more proximal ones. Atherosclerosis in distal arteries in combination with diabetic neuropathy contributes to the higher rates of limb loss in diabetic patients compared with nondiabetic patients (Haid et al., 1970).

Given the limited and severe treatment options presently available for subjects with chronic limb ischemia, it would be desirable to develop methods to improve the perfusion of blood and spare the tissues in the extremities of patients suffering from lower limb ischemia using new therapeutic approaches aimed at promoting the development of new blood vessels in the limbs of these patients.

### SUMMARY OF THE INVENTION

The invention provides methods for treating diseases and disorders associated with limb ischemia that generally comprise administering to a mammal a therapeutically effective amount of a cellular composition comprising multi-potent bone marrow-derived stem cells (BMSC) sufficient to improve circulation and restore adequate levels of oxygenation to the tissues including the limbs. As described below, we have made the unexpected discovery that symptoms of tissue ischemia, especially lower limb ischemia can be effectively treated in this way by administering isolated human multi-potent BMSC directly to tissues and/or a limb in need. The cells are believed to achieve their beneficial effects of restoring blood flow and reducing cell death in ischemic tissues by differentiating into cell types that form new blood vessels and muscle cells when administered into ischemic limb tissue.

The invention thus provides in one aspect a new strategy for preventing, treating, or reducing the severity of disorders involving tissue ischemia, especially limb ischemia. Disorders and conditions that may particularly benefit from the methods and compositions of the invention include atherosclerosis, Buerger's disease, limb ischemia, especially critical limb ischemia, claudication, diabetic neuropathy, chemotherapy induced neuropathy, stroke, transient ischemic attack, Parkinson's disease, and spinal cord injury.

The invention is flexible and can be used alone or in combination with other therapies as needed.

Accordingly, and in one aspect, the invention provides a method for preventing, treating or reducing the severity of tissue ischemia, particularly an ischemic limb disorder, in a mammal having or prone to tissue ischemia. The method involves administering to a host mammal a therapeutically effective amount of a cellular composition or graft comprising an isolated bone marrow-derived stem cell (BMSC). The BMSC population used in the methods of the invention is distinguished from other known bone-marrow derived stem cell populations by having undetectable or low levels of cell markers wherein the cell markers are selected from: CD90, CD117, CD34, CD113, FLK-1, tie-2, Oct 4, GATA-4, NKx2.5, Rex-1, CD105, CD117, CD133, MHC class I receptor and MHC class II receptor, as determined by standard cell marker detection assay (see, e.g., WO2005/042723, especially FIG. 1C; and Example 3 herein).

The method can be practiced on a host mammal including a human patient having an ischemic disorder, particularly an ischemic limb disorder. Some embodiments of the method involve direct administration of the cellular composition or graft to a limb of the host at or near a site impacted by ischemia.

Disorders that will potentially benefit by the method includes those having at least one symptom such as decreased peripheral blood flow, increased apoptosis of cells, and necrosis in the limb tissues, relative to a normal subject.

A particularly advantageous feature of the method is that the BMSC can be derived from the host and expanded in vitro prior to administration back into the host (i.e., allogenic). This aspect provides the potential for cellular therapy using a patient's own cells, thereby avoiding complications associated with immune rejection and immunosuppressive regimens.

In another aspect, the invention features a method for inducing functional new blood vessels in a tissue, particularly a limb of a mammal. The method includes administering to a limb of said mammal a therapeutically effective amount of a cellular composition or graft comprising an isolated bone marrow derived stem cell (BMSC), said cell having undetectable or low levels of the markers discussed above. Such therapies can be useful for those undergoing a procedure (e.g., organ transplant), receiving therapies that can result in ischemia (e.g., chemotherapy), or having a condition that makes an individual prone to ischemia (e.g., diabetes). In a preferred embodiment, cells are obtained from the eventual recipient (i.e., autologous transplant). In such cases, bone marrow cells can be extracted from the subject prior to undergoing the procedure or receiving therapy, or preferably before the development of or at an early stage of ischemia. The BMSCs of the invention are isolated from the bone marrow of the subject, expanded, and delivered to the subject to the site of potential or known ischemia. In the case of organ transplant, the delivery would be preferably be concurrent with or after the transplant is performed.

The invention further includes providing BMSC from a non-host to a host (i.e., allogenic transplant). Ischemia is not always a foreseeable event and can occur due to injury. Moreover, individuals having or prone to ischemia may not be sufficiently healthy to undergo bone marrow donation, or wait for the selection and expansion process to be completed. The invention is not limited by the source of the BMSCs.

The invention further includes identification of individuals having or prone to tissue ischemia, subjecting the individual to at least one of the methods of the invention, and preferably monitoring the individual for prevention, alleviation, or treatment of tissue ischemia. Individuals having or prone to ischemia are those suffering from diseases or conditions associated with poor circulation including, but not limited to, atherosclerosis, Buerger's disease, limb ischemia, particularly critical limb ischemia, claudication, diabetic neuropathy, chemotherapy-induced neuropathy, stroke, transient ischemic attack, Parkinson's disease, tissue transplant, and spinal cord injury.

These and other objects, advantages, and features of the invention will become apparent to those persons skilled in the art upon reading the details of the invention as more fully described below.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It is also to be noted that as used herein and in the appended claims, the singular forms “a”, “an”, and “the” include plural referents unless the context clearly dictates otherwise. Thus, for example, reference to “a disease” includes a plurality of such diseases and reference to “the method” includes reference to one or more methods and equivalents thereof known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

### OVERVIEW OF THE INVENTION

The invention pertains to the therapeutic uses of cellular compositions comprising isolated bone marrow-derived stem cells (BMSC). The BMSCs of the invention have a wide spectrum of important uses including use in the prevention, treatment or alleviation of symptoms associated with vascular disorders, and particularly those directly or indirectly associated with ischemia of the extremities, such as chronic critical limb ischemia and related indications.

As discussed, typical invention methods include administering to a mammal a therapeutically effective amount of a cellular composition of the invention. Sometimes but not exclusively, the method will involve administering a therapeutically effective amount of a cellular composition or graft comprising an isolated human bone marrow cell to a patient in need of such treatment.

### Isolated Bone Marrow-Derived Stem Cells

The invention features an isolated population of novel and multi-potent human BMSC (hBMSC) that have undetectable or low (negligible) levels of at least one and preferably all of the following cell markers: CD90, CD117, CD34, CD113, FLK-1, tie-2, Oct 4, GATA-4, NKx2.5, Rex-1, CD105, CD117, CD133, MHC class I receptor and MHC class II receptor. An “undetectable or low (negligible) level” of a marker is understood as that within a population of BMSC, preferably derived by the methods taught herein, with less than about 3%, preferably less than about 1% of the cells in the population express at least one of the markers listed above. Such markers can be readily detected by what is referred to herein as a standard cell marker detection assay (see, e.g., FIG. 1C of WO 2005/042723). By the phrase “standard cell marker detection assay” is meant a conventional immunological or molecular assay formatted to detect and optionally quantitate one of the foregoing cell markers (i.e., CD90, CD117, CD34 etc.).

Examples of such conventional immunological assays include Western blotting, fluorescence activated cell sorting (FACS), enzyme-linked immunofluorescence assay (ELISA), and radio immunoassay (RIA). Preferred antibodies for use in such assays are provided below. See generally, Harlow and Lane in Antibodies: A Laboratory Manual, CSH Publications, N.Y. (1988), for disclosure relating to these and other suitable assays. Particular molecular assays suitable for such use include polymerase chain reaction (PCR) type assays using oligonucleotide primers disclosed herein (see Table 1, for instance). See WO 92/07075 for general disclosure relating to recombinant PCR and related methods. See also Sambrook et al. in Molecular Cloning: A Laboratory Manual (2d ed. 1989); and Ausubel et al., Current Protocols in Molecular Biology, John Wiley & Sons, New York, 1989; for general disclosure relating to recognized immunological and molecular assays that can be used to detect the cell markers.

### Isolation and Expansion of Multi-Potent Bone Marrow-Derived Stem Cells

Bone marrow (BM) is a highly vascular modified connective tissue that occupies the cavities of most bones. One of the well recognized functions of BM is production of new blood cells. Worn blood cells are periodically removed from the circulation and must be replaced. New blood cells arise from cells in the BM known as stem cells or stem/progenitor cells. Bone marrow has long been recognized as a rich source of many types of stem/progenitor cells, and those that give rise to blood cells have been extensively characterized. Under appropriate conditions certain stem/progenitor cells divide and differentiate along recognized pathways to form blood cells, such as those of the erythroid, myeloid, and lymphoid lineages.

Recently there has been increasing appreciation that BM from adult subjects is not restricted to production of new blood cells, but also is a source of multi-potent stem/progenitor cells (BMSC) with potential to give rise to cells that differentiate into many other lineages, including endothelial cells (EC), muscle cells (MC), including cardiac muscle cells of the heart (CMC), and others.

As discussed, the cellular compositions of the invention comprise isolated cells derived from bone marrow. As used herein, the terms “isolated” and “substantially purified,” used interchangeably herein, when used in the context of an “isolated cell,” or population, graft, or pharmaceutical product comprising such cells refers to a cell that is in an environment different from that in which the cell naturally occurs. As used herein, the term “substantially purified” in the context of isolated cells, cell populations, grafts, and the like refers to a cell that is removed from its natural environment and is at least 60% free, preferably 75% free, and most preferably 90% free from other components with which it is naturally associated.

By “isolated,” as it is used herein to refer to BM cells and BM-derived cells in vitro, or in the form of a pharmaceutical product, it is meant that the cells have been separated from bone marrow and other cell substituents that naturally accompany it. Preferably, the BM or BM-derived cells of the invention are at least 80% or 90% to 95% pure (w/w). BM-derived cells having at least 98 to 99% homogeneity (w/w) are most preferred for many pharmaceutical, clinical and research applications. Once substantially purified or isolated, the transgenic cells would be substantially free of unwanted marrow contaminants. Once purified partially or to substantial purity, the transgenic cells are suited for therapeutic or other uses such as those provided herein. Purity can be determined by a variety of standard techniques

As used herein, an isolated “cell derived from bone marrow (BM)” is meant to refer to any cell type that is either 1) directly obtained from the BM of an animal, or 2) the product of a cell that is directly obtained from the BM. Examples of the former type of cells, in particular various characterized stem/progenitor cells in the BM, are further described infra. A “cell derived from bone marrow” can also refer to a cell of the second type described above, for example a progeny cell that arises by division and/or differentiation of a cell type of the BM, for example following transplantation of a BMSC to a site in the body of a subject such as a tissue site in an ischemic limb. As shown herein, cells “derived from bone marrow” can include progeny cells that arise by division and/or differentiation of a “multipotent stem/progenitor cell” of the BM, as further defined below. Examples of such progeny cells include but are not limited to endothelial cells (EC), immature muscle cells (myocytes), and smooth muscle cells (SMC).

As used herein, an “angiogenic factor” is meant to refer to a compound that promotes the formation of new blood vessels (i.e., angiogenesis). Angiogenesis factors that can be used with the invention include, but are not limited to, vascular endothelial growth factor (VEGF), basic fibroblast growth factor (bFGF), hepatocyte growth factor (HGF), angiotensin-1 (Ang-1), stromal derived growth factor (SDF-1α) and insulin-like growth factor (IGF).

As used herein the term “mitogen” means any protein, polypeptide, mutein or portion that is capable of, directly or indirectly, inducing cell growth, preferably growth of the BMSCs used in the methods of the instant invention. Such proteins include, for example, acidic and basic fibroblast growth factors (aFGF and bFGF), vascular endothelial growth factor (VEGF), epidermal growth factor (EGF), transforming growth factor-alpha and -beta (TGF-alpha and TFG-beta), platelet-derived endothelial growth factor (PD-ECGF), platelet-derived growth factor (PDGF), tumor necrosis factor-alpha (TNF-alpha), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxide synthase (NOS). Muteins or fragments of a mitogen may be used as long as they induce or promote EC cell growth. The use of nucleic acids encoding mitogens is also within the scope of the invention. A compound may be both an angiogenic factor and a mitogen.

### Methods for Isolation and Culturing Human BMSC

Disclosed herein are methods that enable those of skill in the art to prepare populations of isolated human BMSC useful for treatment of ischemic limb disorders. For preparation of maximal numbers of BM-derived cells, a whole BM cell isolate, or a culture of whole BM cells may be used. The art is well advanced in the areas of isolation of BM from bones of host subjects, tissue culture methods for propagation of BM cells, and of subpopulations of BM cells enriched in particular lineages, providing many options for selecting a starting population of BM cells to be used in accordance with the invention. For example, whole BM can be cultured. Depending on the purpose, however, in many applications it may be preferable to use an enriched population of BM cells as the starting material. BM cells can be isolated, for example, by flushing the cells from long bones such as the femur or tibia. Human BM cells from donors can also be obtained from commercial sources. Mononuclear cells in the BM can be isolated by gradient centrifugation and cultured.

As discussed, a particularly preferred BMSC of use in the invention is a human “multipotent BMSC,” (hBMSC), which may be prepared as described in Example 1 and is further described in detail in co-pending publication WO2005/042723, herein incorporated by reference in its entirety.

Methods of the invention involve administering a therapeutically effective amount of the above-described cells in the form of a cellular composition or graft. The terms “effective amount,” “therapeutic amount,” “therapeutically effective amount,” and the like are used interchangeably herein to describe a dosage of a composition comprising isolated cells that is sufficient to provide treatment for the disease state being treated, e.g., ischemia due to vascular insufficiency in a tissue, particularly a limb. In general, an effective amount of BMSC is one that is effective to increase the blood flow to a tissue, particularly a limb, impacted by ischemia and to preserve the integrity of the affected tissues and cells, prevent apoptosis and necrosis, and promote neovascularization. The term “increase” is used interchangeably herein with “stimulate” and “promote.”

More particularly, by an “effective amount of bone marrow-derived stem cells (BMSC)” or related phrase as used herein is meant a sufficient number of cells to reduce at least one symptom of limb ischemia, as determined by what is referred to herein as a “standard limb ischemia assay”. A preferred version of that assay includes performing at least one of and preferably all of the following steps:
(a) performing a procedure such as ligation or occlusion of a major artery supplying the limb of an animal subject such as rodent, to cause reduced blood flow to the limb sufficient to induce ischemic changes, in tissues and cells normally supplied by said artery
(b) introducing detectably labeled bone marrow-derived stem cells (BMSC) (e.g., labeled with a fluorescent dye such as DiI) directly into a limb impacted by the ischemia-inducing procedure
(c) allowing the subject to recover from the ischemia-producing procedure for at least about few hours, preferably about a few days or more, more preferably about a few weeks up to about one to two months; and
(d) evaluating the status of ischemia and vascular function of the subject's limb, e.g., by performing blood flow measurements in the limb (e.g., by laser Doppler perfusion imaging); histological studies of limb tissues, including assays to evaluate cell death (apoptosis, necrosis); immunohistochemical studies to detect expression of markers of desirable differentiated cell types (e.g., endothelial cells, myocytes, smooth muscle cells) arising from the introduced BMSC and assays to detect expression of gene transcripts associated with angiogenesis and tissue preservation.

Preferred embodiments of the assay are discussed below in the Examples section.

### Method of Treatment for Limb Ischemia

As already mentioned, one aspect of the invention is method for preventing, treating or reducing the severity of an ischemic limb disorder in a host subject (preferably a mammal) involving administering an effective amount of a cellular composition comprising multi-potent bone marrow-derived stem cells as described above.

The terms “subject” or “individual” or “patient,” or “host” used interchangeably herein, refer to any subject, particularly a mammalian subject, for whom diagnosis or therapy is desired. By the term “mammal” is meant a primate, domesticated or other mammal such as a rodent or rabbit. A preferred primate is a chimpanzee, monkey or a human patient in need of treatment. Suitable domesticated animals include gerbils, horses, dogs, cats, goats, sheep, pigs, chickens and the like. A preferred rodent is a rat or mouse. A preferred BMSC is isolated from a primate and particularly a human subject such as those in need of therapy for an ischemic limb disorder.

By the phrase “ischemic tissue” is meant damaged tissue having a deficiency in oxygen (also termed “hypoxia”) that is due to vascular disorders, such as narrowing or occlusion of an artery that supplies oxygenated blood to the tissue. It is noted that a limb is comprised of tissue. Vascular disorders result in a deficiency in blood or blood vessels and can cause ischemia at any one of a number of sites including, but not limited to, cerebrovascular ischemia (e.g., stroke), renal ischemia, limb ischemia (due to a circulatory disorder or limb reattachment), and organ ischemia (e.g., a transplanted organ). An individual in need of prevention, alleviation, and/or treatment of ischemia is prone to, suspected of having, or known to have tissue ischemic conditions such as those listed above. For example, individuals with circulatory problems due to organ transplant, chemotherapy treatments, diabetes, or other conditions that damage circulation may be prone to or suspected of having ischemic tissue, even if no such tissue has been observed directly. Tissues after organ transplant may also be prone to ischemia. Individuals with cardiovascular and diabetic disease can be prone to ischemia.

By the term “ischemic limb disorder” is meant any disorder or condition that, due to primary or secondary causes, results in insufficient levels of oxygenated blood to be delivered to tissues in the extremities (arms or legs) of a mammal. Typically, ischemic conditions in a tissue (also termed “hypoxia”) are due to vascular disorders, such as narrowing or occlusion of an artery that supplies oxygenated blood to the tissue. Ischemic limb disorders are associated with many pathological conditions and disorders, including but not limited to atheroslerosis, Buerger's disease, critical limb ischemia, claudication, diabetic neuropathy, chemotherapy-induced neuropathy, stroke, transient ischemic attack, Parkinson's disease, and spinal cord injury.

The terms “treatment,” “treating,” “therapy,” “reducing the severity of . . . ” and the like are used herein to generally refer to obtaining a desired therapeutic, pharmacologic or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, e.g., a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of symptoms of the disease.

The Examples provide general guidance for effective amounts of stem cells to be used for treatment in animal subjects such as mice. Those skilled in the art will readily be able to determine effective amounts for use in human subjects, given the guidance in the Examples (see, for instance, Example 11). The amount of BMSC administered will, of course, be dependent on the size, sex and weight of the subject, the nature and severity of the disease or condition, the manner and schedule of administration, the likelihood of recurrence of the disease, and the judgment of the prescribing physician. Typical amounts of BMSC to use will depend on these and other recognized parameters; however for most applications between from about 10³ to about 10⁷ BMSC will suffice, typically about 10⁵ such cells. Cells may be administered by any acceptable route including suspending the cells in saline and administering same with a needle, catheter or like device. In embodiments in which limb ischemia is to be addressed, the administration may be a bolus injection, for example by injection, preferably intramusclar injection, near or directly into the site of ischemic injury. Cells can also be injected into vasculature at a site proximal to the ischemic tissue. Such methods are known to those skilled in the art (see, e.g., WO 2005/042723)

In testing the compositions and methods of the invention, humans and other species may be used in standard assays. As discussed, suitable mammalian species include, but are not limited to, mice, rats, pigs, rabbits, sheep, dogs and chickens. Test cellular compositions are administered to these animals according to standard methods as described below.

Suitable animal models (further described infra) for induction of limb ischemia and evaluation of treatment modalities involving cell-based therapies include those that are experimentally produced, such as by ligation of an artery of a limb, or other models of naturally occurring or experimentally induced ischemia.

If desired, the method disclosed herein can be used alone or in combination with other recognized therapies that promote angiogenesis in host subjects in need of such treatment. For example, in one embodiment, the method further includes administering to the mammal in need of treatment at least one angiogenic factor and/or mitogen (or functional fragment of the factor or mitogen). Preferred angiogenic factors and/or mitogens (and methods of use) are disclosed in U.S. Pat. No. 5,980,887 and WO 99/45775. Alternatively, or in addition, the method can include administering to the mammal at least one nucleic acid (e.g., by direct injection of a plasmid encoding the mitogen or angiogenic factor directly into tissue, by infusion into vasculature proximal to the ischemic tissue) encoding at least one angiogenic factor or functional fragment thereof. Methods for administering such nucleic acids to the mammal have been disclosed by U.S. Pat. No. 5,980,887 and WO 99/45775, for instance. Treatment with the angiogenic factor/mitogen protein, or nucleic acid encoding same, can precede use of the BMSC or can be administered during or after such treatment as needed.

In yet another embodiment, the method further includes administering to the mammal endothelial progenitor cells (EPC). This invention embodiment especially finds use where good vascular growth is needed to address a vascular disorder. Methods for making and using EPC have been disclosed. See U.S. Pat. No. 5,980,887, for example. Typical methods can include isolating the EPC from the mammal and contacting the EPC with at least one angiogenic factor and/or mitogen ex vivo.

The BMSC-based cellular compositions of the invention are most preferably applied in the form of appropriate pharmaceutical formulations. The pharmaceutically acceptable carrier should be substantially inert, so as not to act with the cells or other active components. “Pharmaceutically acceptable carriers” for therapeutic use are well known in the pharmaceutical arts, and are described, for example, in Remington's Pharmaceutical Sciences, Mack Publishing Co. (A. R. Gennaro, editor, 1985).

It has been further discovered that another beneficial effect of delivery of a cellular composition comprising BMSC to a limb impacted with an ischemic disorder such as lower limb ischemia includes promotion of new blood vessel growth in the ischemic limb. The new vasculature is functional, as evidenced by laser Doppler perfusion imaging showing significantly improved blood flow in the limb. Studies performed on an experimental model of lower limb ischemia treated with human BMSC, described in Examples below (summarized in Tables 2 and 3, infra), show that restoration of the blood flow to the limb is accompanied by a marked improvement in ischemia-induced loss of limbs, and development of new capillaries, accompanied by upregulated expression of a number of angiogenesis-promoting factors by cells in the ischemic limbs transplanted with BMSC of the invention.

Accordingly, yet other aspects of the invention include a method for increasing production of at least one angiogenic factor in an ischemic tissue and/or limb of a mammal in need of such treatment, a method for inducing functional new blood vessels in the tissue and/or limb of a mammal, and method for decreasing apoptosis due to ischemia in the tissue and/or limb of a mammal. These methods each comprise administering a therapeutically effective amount of a cellular composition of the invention, as further described in Examples provided below.

### EXAMPLES

The following Examples are set forth to provide those of ordinary skill in the art with a complete disclosure and description of how to make and use the present invention, and are not intended to limit the scope of what the inventors regard as their invention. Neither are they intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, and temperature is in degrees Celsius

Examples 1-9 below describe materials and methods that may be used in the practice of the invention and/or are demonstrated in studies described in Examples 10 and higher, infra.

### Example 1

### Experimental Animals

Some experiments are performed using a model of limb ischemia induced by ligation of a major artery providing the blood supply to a limb. A useful example of such a model is a rodent (rat or mouse) model of hindlimb ischemia (HLI) produced by ligating a femoral artery (which provides blood supply to the hind limb). This model exhibits pathological features common to limb ischemia in humans, and is characterized, inter alia, by reduced blood flow to the limb which may be assessed in the living animals by laser Doppler perfusion imaging, as well as by apoptosis and necrosis of affected tissues and if continued, ultimate loss of the affected limb.

For experimental paradigms involving transplantation of heterologous stem cells into recipient rodent hosts (such as human BMSC into mouse tissues), a useful host strain of mouse is a nude mouse. This type of laboratory mouse is hairless, lacks a normal thymus gland, and has a defective immune system because of a genetic mutation. Athymic, nude mice are useful for testing procedures involving transplantation of stem cells because they do not reject cells from other mouse strains, or from other species such as humans.

### Example 2

### Isolation and Culture of Bone Marrow Derived Stem Cells (BMSC)

BM cells are isolated from the long bones of a mammal (for example rodents) or from human bone marrow aspirates. Cells are plated on plastic culture dishes and subsequently cultured under suitable conditions. Human BM cells may be isolated and cultured, for example, in phenol red-free EC basal medium EBM-2 (Clonetics) supplemented with 5% fetal bovine serum (FBS), antibiotics and growth factors (EPC medium) on surfaces coated with rat plasma vitronectin (Sigma) in 0.5% gelatin solution.

Particulars of a method for generating a preferred human multipotent bone marrow stem cell (hBMSC) population are further set forth below:

Fresh unprocessed human BM is obtained by standard procedures from a patient, or from a donor, or, depending upon the purpose, may be purchased from commercial sources, e.g., Cambrex Corp. BM is centrifuged at about 350 g for about 10 minutes to obtain cell pellets, which are resuspended in 25 ml of Dulbecco's PBS (DPBS) containing 0.5 M EDTA. After centrifugation at 350 g for about 7 minutes, the cells are resuspended in 5 ml DPBS containing 0.5 M EDTA and 20 ml of NH₄Cl for induction of hemolysis. After centrifugation and washing with DPBS containing 0.5 M EDTA, cells are filtered through a 40-μm nylon filter and plated in wells of 6-well plates that are coated with fibronectin (100 μg/ml).

The cells are grown at a density of about 5×10⁶ per square centimeter in complete DMEM with low (1 g/L) glucose containing about 17% FBS (lot selected for promoting expansion of marrow cells; Cambrex Corp.), 100 U/ml penicillin, and 100 μg/ml streptomycin and 2 mM glutamate at 37° C. and 5% CO₂.

Once the attached cells begin to form colonies (4-6 days), the medium is replaced and the adherent cells are grown to about 60% confluence. Next, the cells are reseeded in complete medium into 25-cm² tissue culture flasks at a density of 1×10⁴ cells per square centimeter. After reaching about 60% confluence, the cells are serially reseeded into 75-cm² and 175-cm² flasks at the same density. After at least 2 passages of culture in 175-cm² flasks, cells are labeled with CellTracker CM-DiI (DiI; Invitrogen Corp.), plated into the wells of a 96-well plate at a density of 0.5 cell per well by limiting-dilution method, and cultured with conditioned media, which are collected prior to limiting dilution, stored in −80° C., and filtered through a 0.22-μm sterile filter (Fisher Scientific International Inc.). After exclusion of wells containing more than 1 cell under fluorescent microscopy, clones derived from a single cell are further cultured.

When these cells are grown to 40-50% confluence, cells from one well are reseeded into one well of a 6-well plate and thereafter serially reseeded in 25-cm², 75-cm², and 175-cm² flasks when cells reach about 30% confluence. When the cells reach a density of 4×10³ to 8×10³ cells per square centimeter in 175-cm² flasks, they are replated at 1:40 to 1:10 dilution; and this process is repeated up to through about 140 population doublings (PD). All reseeding is performed in triplicate, and the fastest-growing clone is selected, e.g., through 50-60 PD.

### Example 3

### Fluorescence-Activated Cell Sorting (FACS) of BMSC Markers

FACS analysis of BMSC such as hBMSCs is performed on cultured hBMSCs. Specimens are selected from at least 3 different populations of clonal lines, and for each clonal line, two sets of cells at different passages (e.g., 5 PDs and 120 PDs are used. The procedure of FACS staining has been described (Kalka C, et al. Proc. Natl. Acad. Sci. U.S.A. 2000; 97:3422-3427). In brief, a total of about 2×10⁵ cultured cells are resuspended with 200 μl of Dulbecco's PBS (Cambrex Corp.) containing 10% FBS and 0.01% NaN₃ and incubated for 30 minutes at 4° C. with directly PE- or FITC-conjugated mAbs or nonconjugated Abs followed by a FITC-conjugated rabbit antimouse IgG (Jackson immunoResearch Laboratories Inc.). Proper isotype-identical Igs serve as controls.

After staining, the cells are fixed in 2% paraformaldehyde, and quantitative FACS is performed on a flow cytometer (FACStar, BD). Antibodies (Abs) suitable for FACS analysis of these cells include: FITC- or PE-conjugated Abs against CD4, CD8, CD11b (Mac-1), CD13, CD14, CD15, CD29, CD30, CD31, CD34, CD44, CD49e, CD71, CD73, CD90 (Thy1), CD117 (c-kit), CD146, CD166, HLA-DR, HLA-ABC, #2-microglobulin (all from BD), CD133 (AC133; Miltenyi Biotec Inc.), and CD105 (endoglin; Ancell Corp.), unconjugated Abs against Oct4 (Santa Cruz Biotechnology Inc.), and isotype control Igs (BD). For comparison, human mesenchymal stem cells (PT-2501) and media (PT-3001) are available e.g., from Cambrex Corp. and are used for FACS.

### Example 4

### DNA Ploidy Analysis, Telomere Length and Telomerase Assay

Ploidy. DNA content per cell is determined by pretreatment of cells with ribonuclease (100 μg/ml), staining with propidium iodide (50 μg/ml), and subsequent FACS analysis.

Telomere length. A TeloTAGGG telomere length assay kit (e.g., from Roche Diagnostics Corp.) can be used for determination of mean telomere length of hBMSCs at 5 PDs and 120 PDs from different clones. Briefly, after isolation (1 μg) and digestion of genomic DNA, DNA fragments are separated by gel electrophoresis and transferred to a nylon membrane by Southern blotting. The blotted DNA fragments are hybridized to a digoxigenin-labeled probe specific for TRF and incubated with a digoxigenin-specific Ab covalently coupled to alkaline phosphate. Finally, the immobilized telomere probe is visualized by alkaline phosphatase metabolizing CDP-Star™, a highly sensitive chemiluminescence substrate. The average TRF length is determined by comparison of the signals relative to a molecular weight standard.

Telomerase activity. A telomeric repeat amplification protocol (TRAP) assay can be performed telomerase activity in hBMSCs with a TeloTAGGG telomerase PCR kit (Roche Diagnostics Corp.).

### Example 5

### Antibodies Useful for Immunocytochemistry

In some procedures it is useful to detect markers of particular cell lineages by fluorescence immunocytochemistry. Procedures for processing cells and tissues and sources of some Abs useful for the purpose are listed below.

Cultured cells are typically fixed in 4% cold paraformaldehyde (PFA) for about 7 minutes and washed with PBS twice. Tissue samples (e.g., from ischemic limbs of experimental subjects) are typically fixed in blocks for processing in frozen or paraffin-embedded sections, and sectioned prior to immunostaining. For frozen sections, OCT embedded frozen sections are typically used.

For markers of endothelial cells (EC), Abs against the following proteins may be used at the indicated dilutions: vWF (goat pAb) (1:400, Sigma, St Louis, Mo.), Flk-1 (mouse mAb) (1:300, Santa-Cruz, Santa-Cruz, C A) VE-Cadherin (mouse mAb) (1:100, BD), CD31 (mouse mAb) (1:100, BD), UEA-1 lectin (1:200, Vector, Burlingame, Calif.), isolectin B4 (1:200, Vector) and DiI-acetylated LDL (Biomedical Technologies, Stoughton, Mass.).

As markers of smooth muscle cells (SMC), useful Abs include those against α-smooth muscle actin (mouse mAb) (1:300), and calponin (mouse mAb) (1:250, DAKO, Carpinteria, C A, USA).

As neural lineage markers, Abs against NF-200 (mouse mAb) (1:400, Sigma), β-tubulinIII (mouse mAb) (1:100, Sigma), Gal-C (rabbit pAb) (1:100, Sigma), and GFAP (goat pAb) (1:200, Santa-Cruz) may be used.

As endodermal (hepatocytic or other epithelial) lineage markers, the following are useful Abs: CK18 (mouse mAb; 1:300) (Sigma), α-fetoprotein (goat pAb, 1:200) (Santa-Cruz), albumin (mouse mAb, 1:400) (Sigma), HNF3β (goat pAb, 1:100) (Santa-Cruz), HNF1α (rabbit pAb, 1:200) (Santa-Cruz).

As cardiac muscle cell (CMC) markers, Abs against cTn I (two forms: mouse mAb and rabbit pAb, 1:100) (Chemicon, Temecular, C A), ventricular myosin heavy chain α (α-MHC) (mouse mAb, 1:100) (Chemicon), α-sarcomeric actinin (clone EA-53, mouse mAb, 1:200) (Sigma), ANP (rabbit pAb) (Chemicon) are useful.

Control mouse, rabbit or goat IgG Abs may be obtained, e.g., from Sigma. Secondary anti-mouse, rabbit or goat antibodies (AMCA, 1:150, FITC or Cy-2, 1:200; Cy-3, 1:200) are available, e.g., from Jackson Immunoresearch (West Grove, Pa.).

### Example 6

### Quantitative RT-PCR

In various experimental settings, it is useful to measure the level of expression of transcripts, for example various factors related to angiogenesis. These transcripts are evaluated, for example, in RNA extracted from limbs of animal subjects administered with a therapeutic dose of BMSC or control cells such as total bone marrow cells (TBMC), or sham injected with vehicle solution alone.

For in vivo studies, tissue samples from normal and ischemic limbs, and optionally those of other tissues are harvested at appropriate intervals following administration of the cells (for example, 1, 2, 3, 5, and 7 days, and 2, 3, 4, 5 and 6 weeks or more), and homogenized in an RNA extraction medium (for example, RNA-Stat (Tel-Test Inc.). RNA is isolated according to the manufacturer's instructions.

For in vitro studies, cells are plated under suitable conditions, as described above. Cells are harvested and RNA is extracted, for example using RNA-Stat™ as described above. Alternatively, total RNA is extracted from cultured cells or from tissues using RNaqueous kit (Ambion, Austin, Tex., USA) according to the manufacturer's protocol.

Total RNA is reverse transcribed, for example using iScript cDNA Synthesis Kit (Bio Rad) and PCR amplification is performed according to standard procedures, for example on a Taqman 7300 instrument (Applied Biosystems) In a typical procedure, one microgram of total RNA is reverse transcribed using random hexamer and Moloney murine leukemia virus reverse transcriptase (Superscript II kit, Roche). The RT product is subjected to PCR, e.g., using Advantage cDNA polymerase mix (Clontech) or Taq polymerase (Roche). For semiquantitative RT-PCR, quantification of mRNA expression of each gene is calculated based on the GAPDH expression.

Useful PCR primers are described in Table 1. Generally suitable PCR conditions are as follows: each sample may contain about 1 μl cDNA in a bout a 20 μl total reaction volume, for example using Platinum Quantitative PCR Supermix-UDG (Invitrogen). Samples may be suitably cycled, e.g., as follows: hold for 2 min at 50° C., and 10 min at 95° C. followed by 2 step PCR for 40 cycles of 95° C. for 15 seconds and 60° C. for 60 seconds. It will be appreciated by those of skill in the art that other conditions may be preferable for amplification of certain transcripts using particular sets of primers.

Suitable primer sequences for amplification of the selected transcripts and probe sequences may be used as follows:

TABLE 1
RT-PCR Primer Sequences and Expected Product Size

| | | | |
|---|---|---|---|
| | | SEQ | |
| | | ID | Product |
| Primer | Sequence | NO: | Size(bp) |

| | | | |
|---|---|---|---|
| Human | | | |
| Oct4-f | 5′-GAG AAC AAT GAG AAC CTT CAG GAG A-3′ | 1 | 219 |
| Oct4-r | 5′-TTC TGG CGC CGG TTA CAG AAC CA-3′ | 2 | |
| CD34-f | 5′-ACC ACT TCC CTC ATC TCT CCT CCA A-3′ | 3 | 421 |
| CD34-r | 5′-AGG GTG AGG GAG GCA GAG ACA GAA A-3′ | 4 | |
| KDR-f | 5′-TGC AGG ACC AAG GAG ACT ATG T-3′ | 5 | 458 |
| KDR-r | 5′-TAG GAT GAT GAC AAG AAG TAG CC-3′ | 6 | |
| Tie2-f | 5′-ATC CCA TTT GCA AAG CTT CTG GCT GGC-3′ | 7 | 512 |
| Tie2-r | 5′- TGT GAA GCG TCT CAC AGG TCC AGG | 8 | |
| | ATG-3′ | | |
| CD31-f | 5′-AGG TCA GCA GCA TCG TGG TCA ACA T-3′ | 9 | 387 |
| CD31-r | 5′-GTG GGG TTG TCT TTG AAT ACC GCA G-3′ | 10 | |
| VE-cadherin-f | 5′-CTC TGC ATC CTC ACC ATC ACA G-3′ | 11 | 389 |
| VE-cadherin-r | 5′-TAG CCG TAG ATG TGC AGC GTG T-3′ | 12 | |
| SM1-f | 5′-TAA ACA CCT CCC CAT CTA CTC GG-3′ | 13 | 732 |
| SM1-r | 5′-ATC TCA TCA TCC TGG GCT GCT GG-3′ | 14 | |
| SM22α-f | 5′-CGG CTG GTG GAG TGG ATC ATA G-3′ | 15 | 489 |
| SM22α-r | 5′-CCC TCT GTT GCT GCC CAT CTG A-3′ | 16 | |
| PDGFRb-f | 5′-GCC TTA CCA CAT CCG CTC-3′ | 17 | 443 |
| PDGFRb-r | 5′-TCA CAC TCT TCC GTC ACA TTG C-3′ | 18 | |
| GATA4-f | 5′-AGA-CAT-CGC-ACT-GAC-TGA-GAA-C-3′ | 19 | 475 |
| GATA4-r | 5′-GAC-GGG-TCA-CTA-TCT-GTG-CAA-C-3′ | 20 | |
| Nkx2.5-f | 5′-CTT-CAA-GCC-AGA-GGC-CTA-CG-3′ | 21 | 233 |
| Nkx2.5-r | 5′-CCG-CCT-CTG-TCT-TCT-TCA-GC-3′ | 22 | |
| AFP-f | 5′-TGC AGC CAA AGT GAA GAG GGA AGA-3′ | 23 | 343 |
| AFP-r | 5′-CAT AGC GAG CAG CCC AAA GAA GAA-3′ | 24 | |
| Alb-f | 5′-TGC TTG AAT GTG CTG ATG ACA GGG-3′ | 25 | 181 |
| Alb-r | 5′-AAG GCA AGT CAG CAG GCA TCT CAT C-3′ | 26 | |
| CK18-f | 5′-GTA CTG GTC TCA GCA GAT TGA GGA G-3′ | 27 | 499 |
| CK18-r | 5′-GCT TCT GCT GGC TTA ATG CCT CAG A-3′ | 28 | |
| CK19-f | 5′-ATG GCC GAG CAG AAC CGG AA-3′ | 29 | 318 |
| CK19-r | 5′-CCA TGA GCC GCT GGT ACT CC-3′ | 30 | |
| GFAP-f | 5′-TCA TCG CTC AGG AGG TCC TT-3′ | 31 | 383 |
| GFAP-r | 5′-CTG TTG CCA GAG ATG GAG GTT-3′ | 32 | |
| MAP2-f | 5′-GAA GAC TCG CAT CCG AAT GG-3′ | 33 | 527 |
| MAP2-r | 5′-CGC AGG ATA GGA GGA AGA GAC T-3′ | 34 | |
| MBP-f | 5′-TTAGCT GAATTC GCG TGT GG-3′ | 34 | 374 |
| MBP-r | 5′-GAG GAAGTGAAT GAG CCG GTTA-3′ | 36 | |
| GAD-f | 5′-GCG CCA TAT CCA ACA GTG ACA G-3′ | 37 | 284 |
| GAD-r | 5′-GCC AGC AGT TGC ATT GAC ATAA-3′ | 38 | |
| Tau-f | 5′-GTA AAA GCA AAG ACG GGA CTG G-3′, | 39 | 512/612 |
| Tau-r | 5′-ATG ATG GAT GTT GCC TAA TGA G-3′ | 40 | |
| Rat | | | 434, 564, |
| VEGF-f | 5′-GGA CCC TGA CTT TAC TGC TGT ACC-3′ | 41 | 631 |
| VEGF-r | 5′-CCG AAA CCC TGA GGA GGC TCC-3′ | 42 | |
| bFGF-f | 5′-TCT ACT GCA AGA ACG GCG GCT TCT T-3′ | 43 | 287 |
| bFGF-r | 5′-CAG TGC CAC ATA CCA ACT GGA GTA T-3′ | 44 | |
| PDGF-B-f | 5′-CCG AGG AGC TTT ATG AGA TGC TGA G-3′ | 45 | 479 |
| PDGF-B-r | 5′-AGC TGC CAC TGT CTC ACA CTT GCA T-3′ | 46 | |
| Nkx2.5-f | 5′-CAG TGG AGC TGG ACA AAG CC-3′ | 47 | 216 |
| Nkx2.5-r | 5′-TAG CGA CGG TTC TGG AAC CA-3′ | 48 | |
| GATA4-f | 5′-CTG TCA TCT CAC TAT GGG CA-3′ | 49 | 275 |
| GATA4-r | 5′-CCA AGT CCG AGC AGG AAT TT-3′ | 50 | |
| MEF2c-f | 5′-AGC AAG AAT ACG ATG CCA TC-3′ | 51 | 407, 311 |
| MEF2c-r | 5′-GAA GGG GTG GTG GTA CGG TC-3′ | 52 | |
| Ang-1-f | 5′-AGT CGG AGA TGG CCC AGA TAC AAC A-3′ | 53 | 169 |
| Ang-1-r | 5′-TCC AGC AGT TGG ATT TCA AGA CGG G-3′ | 54 | |
| Ang-2-f | 5′-TAC GTG CTG AAG ATC CAG CTG AAG G-3′ | 55 | 259 |
| Ang-2-r | 5′-AGT TGG AAG GAC CAC ATG CGT CGA A-3′ | 56 | |
| HGF-f | 5′-CCA ACA CAA ACA ACA GA GGG TGG A-3′ | 57 | 593 |
| HGF-r | 5′-CGA CCA GGA ACA ATG ACA CCA AGA A-3′ | 58 | |
| TGF-β-f | 5′-CAA CTA CTG CTT CAG CTC CAC AGA G-3′ | 59 | 314 |
| TGF-β-r | 5′-AGG AGC GCA CGA TCA TGT TGG ACA A-3′ | 60 | |
| GAPDH-f | 5′-TCG GTG TGA ACG GAT TTG GCC GTA T-3′ | 61 | 505 |
| GAPDH-r | 5′-AGC CCT TCC ACG ATG CCA AAG TTG T-3′ | 62 | |
| SDF-1α-f | 5′-ATG GGA CGC CAA GGT CGT CG-3′ | 63 | 223 |
| SDF-1α-r | 5′-TCG GGT CAA TGC ACA CTT GTC TGT-3′ | 64 | |

-f: forward primer; -r: reverse primer

RT-PCR products are analyzed by 1.5% agarose gel electrophoresis with a 100-bp ladder (Life Technologies) and quantified, e.g., with the UV imager Eagle-Eye II (Stratagene).

Relative mRNA expression of target genes may be calculated, for example, with the comparative C_{T} method. The amount of target genes can be normalized to the endogenous 18S control gene (Applied Biosystems). Difference in C_{T} values is calculated for each mRNA by taking the mean C_{T} of duplicate reactions and subtracting the mean C_{T} of duplicate reactions for 18S RNA. The fold change in expression of the target gene from cells treated relative to control cells is calculated as follows: relative expression=2^{ΔCT}.

### Example 7

### Morphometric Analysis of Blood Vessels and Capillaries

For visualization of blood vessels, immunohistochemical staining can be performed using antibodies prepared against the murine-specific endothelial cell marker isolectin B4 (Vector Laboratories). Capillaries are recognized by fluorescence microscopy as fluorescently labeled tubular structures positive for isolectin B4. Capillary density may be evaluated morphometrically by examination of randomly selected fields of tissue sections recovered from the limbs of animals receiving BMSC and control cells. Preferably, morphometric studies are performed by at least two examiners who are blinded to the treatment protocol.

Capillary density can also be determined by after staining of samples with mAb against CD31 and H&E, as described previously (Kawamoto, Circulation 107:461-468, 2003). For counting, a total of about 10 visual fields in which a cross-section of capillaries is clearly visible is randomly selected, e.g., in an ischemic area, and the number of capillaries is counted, e.g., under ×200 magnification.

Labeling of functional vessels and endothelial precursor cells (EPC). A procedure for labeling vessels in vivo can be performed by injection of rhodamine-conjugated BS1 lectin before sacrifice. For in vitro labeling of EPC, cultured EPCs are co-stained with acetylated LDL (acLDL)-DiI (Biomedical Technologies) and FITC-conjugated isolectin B4 (Vector Laboratories), both of which are markers characteristic of endothelial lineage (Asahara et al., Science 275:964-7, 1997; Asahara et al., EMBO J. 18:3964-72, 1999).

### Example 8

### BrdU Immunohistochemistry and Determination of Apoptosis by TUNEL Staining

BrdU is detected, e.g., with the sheep anti-BrdU antibody (1:50; Biodesign) followed by Streptavidin-FITC (1:100; Vector). Nuclear counterstaining is performed with DAPI. BrdU positive cells are counted in ischemic areas where scar tissue comprises less than 20% of the visual field (×200 magnification).

In situ labeling of fragmented DNA is performed with the terminal deoxynucleotidyltransferase (TdT)-mediated dUTP-biotin nick end labeling (TUNEL) method, using an in situ cell detection kit (e.g., from Roche) as described (Fujio et al., Circulation 101:660-7, 2000). Tissues sections for TUNEL staining are treated with 20 μg of proteinase K. After washes, sections are incubated in a solution of TdT with a fluorescein dUTP mixture. Sections are then counter-stained with DAPI for localization of nuclei. To determine the proportion of apoptotic nuclei within particular cell types, tissue can counterstained with a mAb against a cell-specific marker. Tissue sections are examined microscopically under ×200 magnification, and a total of about 10 random visual fields in an area of interest is examined. The percentage of apoptotic cells is termed the “apoptotic index.”

### Example 9

### Statistical Procedures

Some results are appropriately expressed as mean±S.E. Statistical significance is evaluated using the unpaired Student's t-test between two means. Multiple comparisons among more than two groups are preferably analyzed by ANOVA. A value of P<0.05 denotes statistical significance. All in vitro experiments are preferably repeated at least in triplicate.

### Example 10

### Characterization of Human Multipotent Bone-Marrow Derived Stem Cells (hBMSC)

This Example describes characteristics of continuous cell lines of multipotent stem cells derived from human bone marrow, designated hBMSC.

Continuous cell lines of hBMSC cells were developed using methods as described in Example 2, supra. The clonality, surface epitopes, euploidy, and proliferation of hBMSCs were evaluated as follows.

Fresh unprocessed human BMs from young male donors were purchased. Three different marrow specimens from three different donors were used for SC cultures. After serial culture of total bone marrow cells (TBMC) in plastic dishes in Dulbecco's modified eagle's medium (DMEM) with low (1 g/L) glucose containing 17% of fetal bovine serum (FBS), cells were labeled with red fluorescent dye, DiI. After limiting dilution (1-2 cells per well in 96 well plate), wells containing a single cell visualized by fluorescent microscopy were selected. Of wells containing a single cell, it was observed that 6±4% (range 2-13%) demonstrated survival and proliferation of cells. When cells were grown to 40-50% confluence, cells from each well (one clone) were reseeded into one well of 6-well plates and thereafter serially reseeded in 25 cm² tissue culture flask (T25), T75 and T175 at a density of 4-8×10³ cells/cm², respectively. Subsequently, cells were cultured at a density of 4-8×10³ cells/cm² in T175 and replated 1: 20-40 dilution. Reseeding was performed in triplicate and the most rapidly growing clones were selected in each culture and expanded in serial cultures. After obtaining more than 10 clones from each bone marrow at passage 6, two clones were selected for continuous cultures.

Morphologically, in comparison with mesenchymal stem cells (MSC), hBMSC are more spherical, are smaller in size (<15 μm in diameter) and exhibit a higher nucleus to cytoplasm ratio. Clonal cell lines derived in this manner underwent more than 140 population doublings (PDs). The doubling time was 38±9 hrs between 20 to 80 PDs.

FACS analysis using multiple cell surface epitopes demonstrated minimal to no expression (0-1%) of CD90 and CD117. hBMSC did not differentiate spontaneously and maintained their phenotype during culture expansion. In contrast, prior to clonal isolation, the cultured hBMSC expressed low levels of CD105, CD90 and CD117, whereas mesenchymal stem cells expressed high levels of CD29, CD44, CD73, CD105 and CD90. Major histocompatibility complex (MHC) class I (ABC) and II (DR) molecules and known hematopoietic stem markers (i.e., CD34, CD133, FLK-1, and Tie2) were not expressed.

Analysis of the marker expression by RT-PCR was negative for Oct4 and Rex-1, which are known markers of embryonic stem cells and multipotent adult progenitor cells (MAPC). These results indicate that hBMSC do not belong to the known classes termed HSCs, MSCs or MAPCs and are immunologically inert.

Mean telomere restriction fragment (TRF) length of hBMSC cultured for 5 PDs was about 17 kilobases (kb); when re-tested after 120 PDs, mean TRF remained unchanged. DNA ploidy (the number of DNA copies) was examined by FACS analysis after staining DNA with propidium iodide. hBMSC cultured for 20 and 140 PDs from 3 different clones demonstrated no evidence for increased ploidy, suggesting that the euploidy is maintained during the culture-expansion.

### Example 11

### Regenerative Effect of hBMSC On Limb Ischemia

This Example demonstrates that direct transplantation of hBMSC can restore peripheral circulation and rescue loss of limb in ischemically jeopardized limbs.

Male nude mice, 8 wks of age underwent ligation of a femoral artery to induce hind limb ischemia (HLI), as described in Example 1. Immediately following surgery, mice were randomly assigned into 3 treatment groups (n=25, each group) and received either 5×10⁵ labeled hBMSC, 5×10⁵ labeled total bone marrow cells (TBMC) or saline (PBS) directly into the ischemic hind limbs. Cell labeling was achieved with a red fluorescent dye, i.e., DiI as described above.

Various studies were performed on the transplanted animals at 1, 2 and 4 weeks following induction of HLI and transplantation of cells or injection of vehicle alone.

The results of these studies are as follows. Strikingly, it was observed that the rate of limb loss at 4 wks was significantly lower in the BMSC group, as shown below in Table 2. Laser Doppler perfusion imaging revealed that the blood flow ratio of ischemic to normal HL was significantly greater in the BMSC transplanted group compared to the controls with a probability of P<0.01 at each time point analyzed, i.e., at 1, 2 and 4 wks after transplantation of hBMSC into the ischemic limbs. Consistent with this finding, analysis of capillary density at four weeks after transplantation demonstrated a significant increase in animals whose ischemic limbs were engrafted with hBMSC (Table 2).

TABLE 2
Effect of Transplantation of Human Bone Marrow Derived Stem Cells
(hBMSC) into Ischemic Limbs

| Feature | Saline | TBMC | hBMSC | Probability |
|---|---|---|---|---|
| Limb loss ¹, % | 73 | 60 | 33 | P < 0.01 |
| Apoptosis ², % | 6.7 ± 1.4 | 5.9 ± 1.3 | 2.1 ± 1.1 | P < 0.05 |
| Capillary | 113 ± 17 | 174 ± 21 | 248 ± 31 | P < 0.01 |
| density/mm² ¹ | | | | |

¹ Measured at 4 wk after transplantation of cells;
² Measured at 1 wk after transplantation

Immunohistochemistry of ischemic hind limb muscles in these animals demonstrated that transplanted hBMSC expressed phenotypic markers of endothelial cells (ECs), smooth muscle cells (SMCs) and myocytes, demonstrating differentiation of the transplanted BMSC into these lineages.

To investigate changes in gene expression associated with the rescue effect, quantitative real-time PCR was performed as described above at two weeks after transplantation using appropriate primers selected from Table 1, supra, to assess the levels of the following transcripts in limbs transplanted with hBMSC vs. those receiving saline: vascular endothelial growth factor (VEGF); basic fibroblast growth factor (bFGF); hepatocyte growth factor (HGF); angiotensin-1 (Ang-1), insulin-like growth factor (IGF) and stromal-derived growth factor (SDF-1α).

The results, summarized in Table 3, reveal that each of these factors was substantially upregulated in the limbs transplanted with hBMSC, at a statistically significant level.

TABLE 3
Effect of Transplantation of hBMSC on Gene Expression of Angiogenic
Factors in Ischemic Limbs

| Transcript | Fold increase | Probability |
|---|---|---|
| VEGF | 3.3 | P < 0.01 |
| BFGF | 1.8 | P < 0.05 |
| HGF | 3.8 | P < 0.01 |
| Ang-1 | 1.5 | P < 0.05 |
| IGF | 2.9 | P < 0.05 |
| SDF-1α | 1.7 | P < 0.05 |

Collectively, the results of studies described in this Example demonstrate that direct transplantation of BMSC effectively restores hindlimb blood flow, preserving tissue integrity in an ischemic HL model through a mechanism that involves cellular differentiation as well as paracrine effects. Accordingly, it is believed that cell-based therapy with hBMSC, a multi-potent adult stem cell, could represent a promising new therapeutic approach for treatment of lower extremity ischemia.

### REFERENCES

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
Ausubel
Brown A L, Juergens J L. Arteriosclerosis and atherosclerosis. In: Allen E V, et al. Peripheral vascular disease. Philadelphia: Saunders, 1972; 57-66.
Cheshire N J, Wolfe J H, Noone M A, Davies L, Drummond M. The economics of femorocrural reconstruction for critical leg ischemia with and without autologous vein. J Vasc Surg 1992; 15:167-74.
Cutajar C L, Marston A, Newcombe J F. Value of cuff occlusion pressures in assessment of peripheral vascular disease. Br Med J 1973; 2(863):392-5.
DeBakey M E, Crawford E S, Garrett E, et al. Occlusive disease of the lower extremities in patients 16 to 37 years of age. Ann Surg 1964; 159:873-90.
Donaldson M C, Mannick J A, Whittemore A D. Femoral-distal bypass with in situ greater saphenous vein. Long-term results using the Mills valvulotome. Ann Surg 1991; 213:457-64.
Asahara T, et al. Isolation of putative progenitor endothelial cells for angiogenesis. Science 1997; 275:964-7.
Asahara T, et al. VEGF contributes to postnatal neovascularization by mobilizing bone marrow-derived endothelial progenitor cells. EMBO J. 1999; 18:3964-72.
Eklund A E, Eriksson G, Olsson A G. A controlled study showing significant short term effect of prostaglandin E1 in healing of ischaemic ulcers of the lower limb in man. Prostaglandins Leukot Med 1982; 8:265-71.
Fujio Y, et al. Akt promotes survival of cardiomyocytes in vitro and protects against ischemia-reperfusion injury in mouse heart. Circulation 2000; 101:660-7.
Gordon T, Kannel W B. Predisposition to atherosclerosis in the head, heart, and legs. The Framingham study. JAMA 1972; 221:661-6.
Haid S P, Conn J Jr, Bergan J J. Cystic adventitial disease of the popliteal artery. Arch Surg 1970; 101:765-70.
Haimovici H. Patterns of arteriosclerotic lesions of the lower extremity. Arch Surg 1967; 95:918-33.
Kalka C, et al. Transplantation of ex vivo expanded endothelial progenitor cells for therapeutic neovascularization. Proc. Natl. Acad. Sci. U.S.A. 2000; 97:3422-3427.
Kawamoto A, et al. Intramyocardial transplantation of autologous endothelial progenitor cells for therapeutic neovascularization of myocardial ischemia. Circulation 2003; 107:461-8.
Mavor G E. The pattern of occlusion in atheroma of the lower limb arteries. The correlation of clinical and arteriographic findings. Br J Surg 1956; 43:352-64.
Ouriel K, McDonnell A E, Metz C E, Zarins C K. Critical evaluation of stress testing in the diagnosis of peripheral vascular disease. Surgery 1982; 91:686-93.
Quinones-Baldrich W J, Prego A A, Ucelay-Gomez R, Freischlag J A, Ahn S S, Baker J D. Long-term results of infrainguinal revascularization with polytetrafluoroethylene: a ten-year experience. J Vasc Surg 1992; 16:209-17.
Santilli J D, Rodnick J E, Santilli S M. Claudication: diagnosis and treatment. Am Fam Physician 1996; 53:1245-53.
Taylor L M Jr, Porter J M. Current status of the reversed saphenous vein graft. In: Bergan J J, Yao J, eds. Arterial surgery. New York: Grune and Stratton, 1987:494-501.
Taylor L M Jr, Edwards J M, Porter J M. Present status of reversed vein bypass grafting: five-year results of a modern series. J Vasc Surg 1990; 11:193-205.
Welch H J. Chronic lower extremity ischemia. Compr Ther 1997; 23:534-8.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for preventing, treating or reducing the severity of tissue ischemia in a mammal having or prone to having ischemic tissue, the method comprising administering to the mammal a therapeutically effective amount of a cellular composition or graft comprising an isolated bone marrow-derived stem cell (BMSC), the cell having an undetectable level of a cell marker selected from the group consisting of: CD90, CD117, CD34, CD113, FLK-1, tie-2, Oct 4, GATA-4, NKx2.5, Rex-1, CD105, CD117, CD133, MHC class I receptor and MHC class II receptor, as determined by a cell marker detection assay.

2. The method of claim 1, wherein the ischemia includes at least one symptom selected from the group consisting of decreased peripheral blood flow, increased apoptosis of cells, and necrosis, relative to a normal subject.

3. The method of claim 1, wherein the ischemia is due to tissue transplantation or chemotherapeutic interventions.

4. The method of claim 1, wherein tissue ischemia is associated with a condition selected from the group consisting of atherosclerosis, Buerger's disease, limb ischemia, claudication, diabetic neuropathy, chemotherapy-induced neuropathy, stroke, transient ischemic attack, Parkinson's disease, and spinal cord injury.

5. A method for inducing a functional new blood vessels in a tissue of a mammal comprising administering to the mammal a therapeutically effective amount of a cellular composition or graft comprising an isolated bone marrow derived stem cell (BMSC), the cell having an undetectable level of a cell marker selected from the group consisting of: CD90, CD117, CD34, CD113, FLK-1, tie-2, Oct 4, GATA-4, NKx2.5, Rex-1, CD105, CD117, CD133, MHC class I receptor and MHC class II receptor, as determined by a cell marker detection assay.

6. A method for decreasing apoptosis due to ischemia in a tissue of a mammal comprising administering a therapeutically effective amount of a cellular composition or graft comprising an isolated bone marrow derived stem cell (BMSC), the cell having an undetectable level of a cell marker selected from the group consisting of: CD90, CD117, CD34, CD113, FLK-1, tie-2, Oct 4, GATA-4, NKx2.5, Rex-1, CD105, CD117, CD133, MHC class I receptor and MEC class II receptor, as determined by a cell marker detection assay.

7. A method for stimulating production of an angiogenic factor in a mammal comprising administering a therapeutically effective amount of a cellular composition or graft comprising an isolated bone marrow derived stem cell (BMSC), the cell having an undetectable level of a cell marker selected from the group consisting of: CD90, CD117, CD34, CD113, FLK-1, tie-2, Oct 4, GATA-4, NKx2.5, Rex-1, CD105, CD117, CD133, MHC class I receptor and MHC class II receptor, as determined by a cell marker detection assay.

8. The method of claim 1, wherein the mammal is a human patient.

9. The method of claim 1, further comprising administering the cellular composition or graft at or near an ischemic site.

10. The method of claim 9, wherein the cellular composition or graft is administered by intramuscular injection.

11. The method of claim 9, wherein the cellular composition or graft is administered into the vasculature proximal to the ischemic site.

12. The method of claims 1-11, wherein undetectable or low expression of a cell marker is less than about 3%.

13. The method of claim 1, further comprising administering of an angiogenic factor to the mammal.

14. The method of claim 13, wherein the angiogenic factor is selected from the group consisting of vascular endothelial growth factor (VEGF), basic fibroblast growth factor (bFGF), hepatocyte growth factor (HGF), angiotensin-1 (Ang-1), stromal derived growth factor (SDF-1α) and insulin-like growth factor (IGF).

15. The method of claim 1, wherein the BMSC is derived from the mammal and expanded in vitro prior to administration to the mammal.

16. The method of claim 1, wherein the BMSC is derived from a non-host and expanded in vitro prior to administration to the mammal.

17. The method of claim 1, wherein the BMSC is treated with a mitogen and/or an angiogenic factor in vitro.
